(19) United States
(12) Patent Application Publication
(10) Pub. No.: US 2014/0287527 A1
(43) Pub. Date: Sep. 25, 2014

(54) METHOD AND APPARATUS FOR TIME-RESOLVED FLUORESCENCE IMMUNOASSAY TESTING

(71) Applicant: LAND AND LONG INTERNATIONAL TRADING CO. LIMITED, Hong Kong (CN)

(72) Inventor: Liwen Xiao, Shanghai (CN)

(73) Assignee: LAND AND LONG INTERNATIONAL TRADING CO. LIMITED, Hong Kong (CN)

(21) Appl. No.: 13/991,472

(22) PCT Filed: Mar. 19, 2013
(86) PCT No.: PCT/CN2013/000317

§ 371 Date: Jun. 4, 2013

(51) Int. Cl. G01N 33/543

(52) U.S. Cl. CPC G01N 33/54306
USPC 436/501; 422/69; 530/391.3

(57) ABSTRACT

A method and apparatus for assaying to detect the presence or quantity of an analyte in a test sample, comprising: forming an unbounded aqueous mixture of a first test sample with a defined amount of a nanosphere probe which is conjugate of an analyte capturing member and a long emission fluorescent label, contacting a contacting zone of a test strip with the aqueous mixture, the test area having bound thereat a test binding moiety that for a competition assay binds any sample in competition with the analyte capturing member; or for a sandwich assay binds any sample analyte non-competitively with the analyte capturing member, the control area having bound a control binding moiety to nanosphere probe, selectively measuring long emission fluorescence at the test and control areas, and for a given test strip, determining a test zone value normalized with the total of test and control area signals.

The invention relates to a method for detecting the presence or quantity of an analyte in a test sample, an assay kit, and a nanosphere probe for use in the assay.

Time-resolved fluorescence assay (time resolved fluoroimmunoassay [sic], TRFIA) is a relatively recent type of detection means. TRFIA employs a rare earth ion as a tracer for labeling proteins, peptides, hormones, antibodies, nucleic acid probes, or biologically active cells. Together with a chelating agent that binds the ion and an enhancement solution (not needed in some cases), the ion is used in the desired reaction system (for example, the antigen-antibody immune response, biotin-avidin reaction, nucleic acid probe hybridization, target-effector cell killer response, and the like). After reaction, the fluorescence intensity in the final product is measured by time-resolved fluorescence, and the concentration of the analyte in the reaction system may be inferred from fluorescence intensity, which may be normalized against control readings. For example, see U.S. Pat. No. 7,632,653. Along with chemiluminescence and electrochemiluminescence immunoassay technology, TRFIA has been labeled one of the top three ultra-sensitive detecting technologies, and enjoys a wide range of applications in food testing, clinical medicine testing, biological research testing, and environmental testing.

As rare earth complexes all have low fluorescence intensities, there is a need to use fluorescence enhancement techniques to improve testing sensitivity. Three categories of TRFIA are currently recognized, distinguished by different signal enhancement techniques: (1) dissociation-enhanced technology (dissociation-enhanced lanthanide fluorescent Immunoassay; DELFIA); (2) CyberFluor system; and (3) nanosphere-based TRFIA (nano-TRFIA). Of these, nano-TRFIA is an entirely new time-resolved fluorescence testing means, which combines the long life of rare earth element fluorescence with the signal amplification effect of nanospheres. Rare earth elements and the coordination complexes thereof are doped together onto nanospheres and microspheres. Following surface activation, antibodies for example labeled with such markers form a complex which, when used for immunoassay, can greatly improve sensitivity and obtain a broader linear range. In practice, actual performance is at least comparable to that of DELFIA technology.

CN02144517 discloses the preparation of highly fluorescent rare earth nanoparticles (Lanthanide Fluorescence Nanoparticles, abbreviated LFNP) and a method for applying same in biological testing technologies. These particles were based on a luminescent center of highly fluorescent rare earth complexes, and prepared via chemical coating with silica gel.

CN03133857 discloses a β-diketone-trivalent europium complex nano-fluorescent probe and the preparation and application thereof. The invention relates to functional rare earth fluorescent nanoparticles prepared from a monomer capable of undergoing copolymerization with a silicate ester, where the monomer undergoes a covalent bonding reaction with a fluorescent trivalent europium-β-diketone complex in an organic solvent, followed by copolymerization with the silicate ester. The molar ratio of trivalent europium ion, β-diketone organic ligand, copolymerizable monomer and silicate ester is 1:2-3:10-100:350-450.

However, existing fluorescent rare earth probes are still hampered by defects such as low fluorescence intensity, low analytical sensitivity, and a tendency for photobleaching. Moreover, there is a need to modify TRFIA methods to provide the speed, ease-of-use and convenience needed for conducting point-of-care analyses.

Also, there is a need for a more accurate, more sensitive, and shelf stable apparatus and method for implementing time-resolved fluorescence immunoassay testing. The assay and assay kits of the invention are unexpectedly more stable, and produce greater sensitivity, than assays in which a conjugate of an analyte capturing member and a fluorescent label are staged on an assay strip.

### SUMMARY OF THE INVENTION

Embodiments of the present invention generally relate to a method and apparatus for improving fluorescence and detection in time-resolved immunoassays as shown in and/or described in connection with at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIGS. 1A to 1C are schematic illustrations of components used in the method of an embodiment of the invention.

FIG. 2 is a diagram of a method for is a schematic representation of molecular components used in the method of an embodiment of the invention.

FIG. 3 is a flow diagram of an exemplary method of implementing an embodiment of the invention.

While the invention is described herein by way of example using several embodiments and illustrative drawings, those skilled in the art will recognize that the invention is not limited to the embodiments of drawing or drawings described. It should be understood that the drawings and detailed description thereto are not intended to limit the invention to the particular form disclosed, but on the contrary, the invention is to cover all modification, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including, but not limited to.

An “unbounded” aqueous mixture is one that is not in the context of a polymer matrix. For example, such a mixture is not that formed by drawing a sample through a staging area of a test strip. Furthermore, it is envisioned the embodiments herein may be used in a point of care setting as well as in a laboratory control setting.

### DETAILED DESCRIPTION

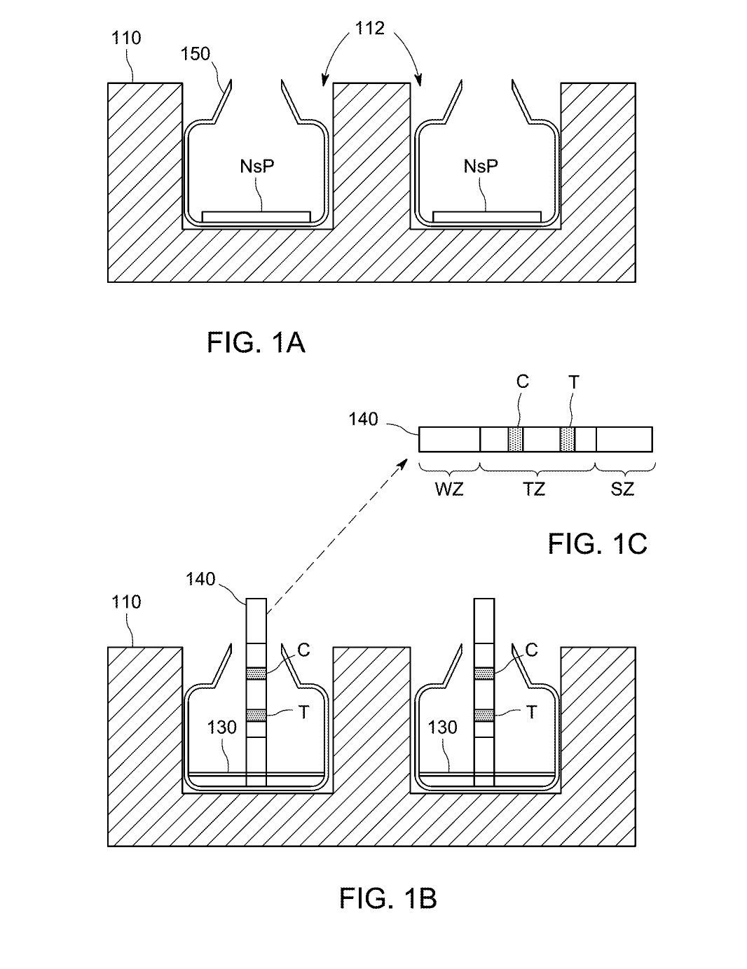

FIG. 1A schematically shows a tray 110 (e.g., heating block, test plate, or the like) with two test wells 112. Vessels 150 (optional) are in the test wells 112. The vessels (e.g., vials, bottles, test tubes, and the like) have within them a defined amount of nanosphere probe NsP in a stabilized form, such as a lyophilizate.

FIG. 1B shows the tray with vessels after the nanosphere probe NsP has been mixed with sample, forming an aqueous mixture 130. A test strip 140 is contacted with the aqueous mixture at a sample zone SZ. The aqueous fluid flows from the sample zone SZ to the test zone TZ. A optional wicking zone WZ can help provide for a greater but regulated flow from the aqueous zone. A test area T has bound thereto the detection binding moiety. A control area C has bound thereto the control binding moiety. FIG. 1C is a blow-up of the test strip 140. Exemplary materials for the sample zone (sample pad), test zone (membrane) and wicking zone (wicking pad) are described in U.S. Pat. No. 7,632,653, the descriptions thereof of test strips and methods of using them are incorporated herein in their entirety.

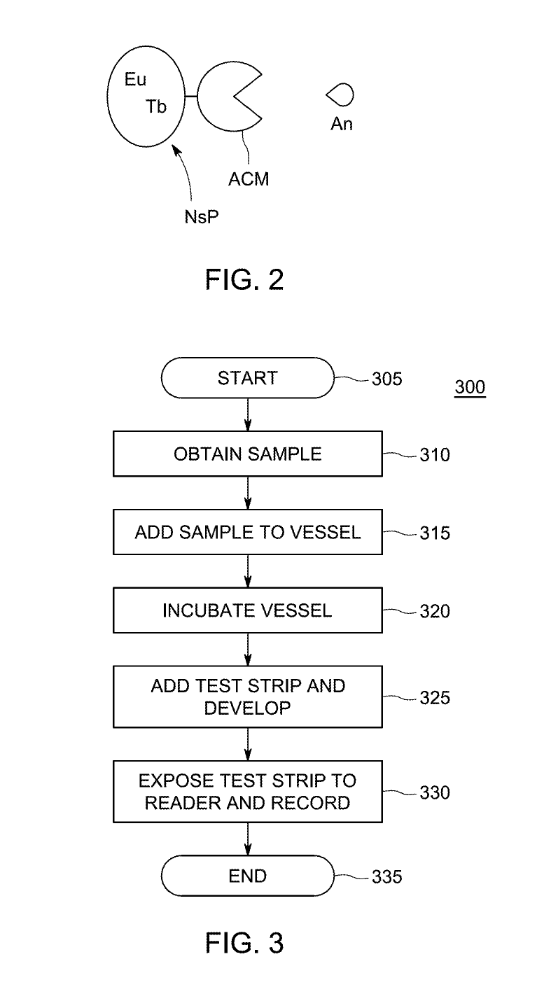

FIG. 2 schematically shows a nanosphere probe NsP and an analyte Art. The nanosphere probe NsP has a rare earth complex (indicated with the illustrative rare earth salts Eu³⁺ and Tb³⁺), and an analyte capturing member (“ACM”, Pac-man-like figure). A schematic analyte An is shown. The analyte An is illustrated as too small to provide useful binding to two separate regions of the molecule (or molecular complex). Thus, the illustrated analyte is appropriate for a competition assay, whereby the signal for greater amounts of analyte correlates with weaker long term fluorescence at the test area T. Larger analytes such as proteins or protein complexes can also be examined with a sandwich assay, in which the ACM binds one domain, and the detection binding moiety binds another. The schematic figure does not imply the ratio of binding members to nanospheres, nor the ratio of rare earths to nanospheres.

In sandwich assays, generally the analyte capturing member (ACM) is an antibody (which can be a chemical derivative of the product of a biological system, or a genetic or chemical mimic of such a product, such as a chimeric antibody) that binds the analyte. The detection binding moiety is a separate antibody that also binds the analyte. The fluorescent signal generated in an assay is proportional to the analyte concentration. The control binding moiety can be antibody that binds to the antibody of the ACM, or can be a component that binds all protein like the antibody of the ACM that reaches the control area C.

For competition assays, the detection binding moiety can be for example a fixed-in-place copy of the analyte or a mimic thereof. For example, for a melamine assay, the ACM can be an antibody, the detection binding moiety can be melamine conjugated (chemically) to ovalbumin or bovine serum albumin, and the control binding moiety can be antibody that binds to the antibody of the ACM. The fluorescent signal generated in a competitive assay is inversely proportional to analyte concentration.

The antibodies can be polyclonal or monoclonal. Polyclonal antibodies are generated by immunizing animals such as rabbits, goats, sheep, etc. The antibodies generated are found in the animals' blood. These antibodies can be used in TRFIA reactions in the form of either serum or plasma. Alternatively, these polyclonal antibodies can be purified by Protein A, Protein G, or affinity methods before use.

Monoclonal antibodies can typically be obtained by immunizing an animal such as a mouse with the desired immunogen. The spleen cells of the mouse are then fused with myeloma cells. The cells producing the desired antibodies are then selected and cloned in order to consistently produce the same antibody. A detailed description of how monoclonal antibodies can be made has been described by Koehler and Milstein. (Koehler, G.; Milstein, C. (1975) “Continuous of cultures of fused cells secreting antibodies of predefined specificity”, Nature 256 (5517): 495-497).

To raise antibodies against macromolecules such as proteins, the immunogen is usually injected into the animals directly after mixing with oily compounds such as Freund's complete or incomplete adjuvants. To generate antibodies against haptens (small molecular weight immunogens), the hapten will have to be chemically conjugated to a carrier protein such as keyhole limpet hemocyanin (KLH), bovine serum albumin, or ovalbumin before it can be injected into animals.

Sandwich assays can be used to detect macromolecules which usually contain more than one epitope (antibody binding site). Thus, at least two antibodies can bind to the same macromolecule at one time. In detecting haptens, competitive assays are commonly used because each hapten typically contains only one epitope making it sterically difficult or impossible for two antibodies to bind to the hapten simultaneously.

The disclosed TRFIA is anticipated to be suitable for detecting a large number of proteins via the sandwich format. These proteins include but are not limited to the following: prostate specific antigen (PSA), human clorionic gonadotropin (HCG), bovine pregnancy specific glycoproteins.

The disclosed TRFIA is also anticipated to be suitable for detecting a large number of haptens via the competitive format. These haptens include but are not limited to the following: antibiotics such as beta-lactams, chloromycetin, tetracyclins, sulfonamides, other drugs such as quinolones, clenbuterol, ractopamine.

A “long emission fluorescent label” is one with an emission lifetime of greater than 1 microsecond. Methods of selectively measuring long emission fluorescence are described for example in U.S. Pat. No. 7,632,653, the descriptions thereof of such measurements are incorporated herein in their entirety.

The nanosphere probe is sufficiently linked to its component parts that the parts remain linked to flow through the test strip, and bind the test area or control area together sufficiently to preserve the function of the assay. The test binding moiety and the control binding moiety are “bound” to the test strip, in that they remain localized and functional sufficiently to preserve the function of the assay. Typically, they are adsorbed at the test area or control area (e.g., via Van der Waals forces), but they can be covalently linked to the test strip.

In conducting the assay, the test samples may have been stored chilled or frozen. Accordingly, it can be useful to incubate the wells that are set up for testing prior to inserting the test strip. For example, the wells can be incubated at 37° C. for 3 minutes.

In certain embodiments, the nanosphere probe comprises Eu³⁺ and another lanthanide, with the other lanthanide in a molar percentage of lanthanide of 0.1% to 10%. In certain embodiments, the other lanthanide is Sm³⁺, Tb³⁺, Nd³⁺, Dy³⁺, or a mixture thereof.

In certain embodiments, the nanospheres have a particle diameter of 10 to 400 nm. In certain embodiments, the nanospheres have a surface charge of 170 to 200 μeq/g. In certain embodiments, the nanospheres have a carboxyl density of 25 to 35.7 sq. Å/grp (parking area).

In certain embodiments, the nanoparticles comprise rare earth ion, β-diketone chelating agent. In certain embodiments, the molar percentage of rare earth ion (exclusive of counter-ion) relative to the total rare earth ion and β-diketone content, is 10 to 30%.

In certain embodiments, the nanoparticles comprise rare earth ion and fluorescence enhancing synergist. In certain embodiments, the molar percentage of fluorescence enhancing synergist relative to the total rare earth ion and fluorescence enhancing synergist content, is 70 to 90%. In certain embodiments, the nanoparticles comprise rare earth ion, β-diketone chelating agent, and fluorescence enhancing synergist in a molar ratio of 1:4:5.

In certain embodiments, the fluorescence enhancing synergist is trioctylphosphine oxide and/or phenanthroline. A fluorescence enhancing synergist is a compound that increases the fluorescent signal from a rare earth fluorophore. In certain embodiments, the assay method of the invention can be conducted within 10 or 15 minutes of when a test sample is available. Where the test sample is blood, the test strip can be adapted to retain red blood cells so that their color does not interfere at the test area or control area. In certain embodiments, blood may be separated (e.g., by centrifugation) to provide a plasma as the test sample.

The nanosphere probe in the vessel is dried to a form that is stable in storage, yet is quickly restored to a functional form when wetted with an appropriate aqueous sample. An appropriate aqueous sample will be recognized (e.g., in terms of pH, salt concentrations, and the like) by those of skill taking consideration of the molecular form of the nanosphere probe.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

### Exemplary Step 1: Preparation of Carboxylated Polystyrene Nanospheres

10 mm of styrene monomer and 0.95 mm acrylic acid monomer were dissolved in 10 mL of deionized water containing 0.45 mm dodecyl sulfonate, and added to a round bottom flask. After stirring evenly with a magnetic stirrer, high purity nitrogen gas was used to purge the round bottom flask of air, before sealing the flask and heating to 70° C. 0.5 mL of 0.15 mm potassium persulfate was added, and allowed to react under stirring in sealed oxygen-free conditions for 8 h. The flask was then cooled to room temperature and the reaction liquid filtered with Whatman 2V filter paper (pore diameter 8 μm). At the end, a dialysis bag (molecular weight cutoff 30,000 Da) was employed to dialyze in deionized water for 5 days, the liquid in the dialysis bag was collected and stored at 4° C. with 0.05% sodium azide.

The diameter of the prepared carboxylated polystyrene nanospheres was measured to be 190±10 nm. The surface charge (μeq/g) was 170 to 200, and the carboxyl density (parking area, sq. Å/grp) 25 to 35.7.

### Exemplary Step 2: Preparation of Fluorescent Nanospheres

10 mL of a mixture of deionized water and acetone (v/v=1:1) was added to a small amount of the 190 nm polystyrene microspheres prepared in Exemplary Step 1, so that the density of polystyrene microspheres in the reaction solution was about 1×1014. After stirring thoroughly, 100 μL of 0.1M europium trichloride, 1 μL of 0.1M terbium trichloride, 400 μL of 0.1M β-dione (β-NTA, β-naphthyl formyl trifluoroacetone, i.e., 2-naphthyloyltrifluoroacetone), 300 μL of trioctylphosphine oxide (TOPO), and 100 μL phenanthroline were added. The mixture was first heated to a constant temperature of 60° C. to undergo dark reaction under stirring for 10 h, and then cooled to room temperature to react for a further 2 h. Finally, the organic solvent in the solution was removed by distillation under reduced pressure, and the solution was dialyzed against deionized water for 5 days to remove the remaining residual small molecules. The liquid in the dialysis bag was collected and stored at 4° C. with 0.05% sodium azide.

Through testing and calculation, the average number of europium ion chelates wrapping each fluorescent nanosphere was found to be about 180,000 to 200,000.

### Exemplary Step 3: Nanosphere Components

Referring to the formulation of Exemplary Step 2, fluorescent nanospheres with (a) no terbium ions, (b) terbium ions and no phenanthroline, and (c) no terbium ions but with phenanthroline were successively prepared, and the fluorescence intensities compared. The results are showed in Table 1.

TABLE 1

| | | | Terbium | No terbium | |
|---|---|---|---|---|---|
| | | No | ions, no | ions, | Commercial |
| Wrapping | | terbium | phenan- | phenan- | fluorescent |
| method | Ex. 2 | ions | throline | throline | microspheres |
| Fluorescence | 180000 | 110000 | 150000⁺ | 130000 | 80000 |
| intensity | | | | | |

In the table above: (1) The fluorescence intensity is defined as the multiple of the fluorescence intensity generated after excitation of one nanosphere to the fluorescence intensity generated by a single free europium chelate; and (2) The commercial fluorescent microspheres had a particle diameter of 0.2 μm, and were purchased from Thermo Fisher Scientific, with the trade name Fluoro-Max Carboxylate-Modified and Streptavidin-Coated Europium Chelate Particles.

Exemplary Step 4: Nanospheres Labeled with Melamine Monoclonal Antibodies

A small amount of the fluorescent nanospheres prepared in Exemplary Step 2 was dissolved in 10 mL of 0.01M pH 8.0 borate buffer to give a density of fluorescent nanospheres of about 1.0×10¹²/mL. Following ultrasonic treatment at 400 W for 30 seconds, the solution was slowly added to 200 μL of 15 mg/mL carbodiimide (1-Ethyl-3-[3-dimethylaminopropyl]carbodiimide hydrochloride, EDC), before incubating at room temperature under uniform stirring for 15 min. Thereafter, centrifugation at 150,000 g was performed for 10 minutes, the precipitate was collected, washed repeatedly with an 0.01M borate buffer of pH 8.0, and then centrifuged twice to obtain activated fluorescent nanospheres. The activated fluorescent nanospheres were redissolved in 5 mL of 0.01 M borate buffer at pH 8.0. 250 μg of melamine monoclonal antibody was added, and allowed to react under agitation for 12 h at 4° C. Centrifugation at 12,000 g was then carried out for 10 minutes, and the precipitate collected and re-dissolved in a 0.01M pH 7.4 phosphate buffer containing 1.5% (m/v) of trehalose and 2% (m/v) bovine serum albumin to yield fluorescent nanosphere labeled melamine monoclonal antibody, which was stored at 4° C. and set aside.

Exemplary Step 5: Nanospheres Labeled with Rabbit IgG

A small amount of the fluorescent nanospheres prepared in Exemplary Step 2 was dissolved in 10 mL of 0.01M pH 8.0 borate buffer to give a density of fluorescent nanospheres of about 1.0×10¹²/mL. Following ultrasonic treatment at 400 W for 30 seconds, the solution was slowly added to 200 μL of 15 mg/mL carbodiimide (EDC), before incubating at room temperature under uniform stirring for 15 min. Thereafter, centrifugation at 150,000 g was performed for 10 minutes, the precipitate was collected, washed repeatedly with an 0.01M borate buffer of pH 8.0, and then centrifuged twice to obtain activated fluorescent nanospheres. The activated fluorescent nanospheres were re-dissolved in 5 mL of 0.01 M borate buffer at pH 8.0. 600 μg of rabbit IgG was added, and allowed to react under agitation for 12 h at 4° C. 12,000 g centrifugation was then carried out for 10 minutes, and the precipitate collected and redissolved in a 0.01 M pH 7.4 phosphate buffer containing 1.5% (m/v) of trehalose and 2% (m/v) bovine serum albumin to yield fluorescent nanosphere-labeled rabbit IgG, which was stored at 4° C. and set aside.

### Exemplary Step 6: Lyophilization of Nanospheres

The nano-fluorescent probes prepared in Exemplary Steps 3 and 4 were respectively diluted 20-fold and 30-fold in a lyophilization diluent (0.05M pH 7.4 PBPS buffer containing 6% sucrose, 4% bovine serum albumin and 1% mannitol), and then mixed thoroughly 1:1 (v/v) before being dispensed in reaction vessels at 100 μL per well. The vessel was lyophilized to dry (see Table 2 for the lyophilization curve), and then sealed with silicone plugs.

TABLE 2

| Temp. | Adjusting time | Holding time | Pressure |
|---|---|---|---|
| −55° C. | 30 min | 240 min | Atmospheric |
| −35° C. | 30 min | 180 min | 0.15 mbar |
| −15° C. | 30 min | 480 min | 0.15 mbar |
| −5° C. | 30 min | 120 min | 0.11 mbar |
| 5° C. | 30 min | 120 min | 0.11 mbar |
| 25° C. | 30 min | 240 min | 0.15 mbar |
| 25° C. | 5 min | 60 min | 0 mbar |

### Exemplary Step 7: Test Strips

1) Nitrocellulose Membrane with C/T Areas

Melamine with ovalbumin conjugate (MEL-OVA) was dissolved to a final concentration of 0.1 mg/mL in a 0.01M pH 7.4 phosphate buffer containing 1.5% (m/v) of trehalose, 2% (m/v) bovine serum albumin, and 0.05% (v/v) Tween −20, and then sprayed with a sprayer at 2 mm in from the left end of the nitrocellulose membrane to form the test (T) line. Goat anti-rabbit secondary antibody was dissolved to a final concentration of 1.0 mg/mL in a 0.01M pH 7.4 phosphate buffer containing 1.5% (m/v) of trehalose, 2% (m/v) bovine serum albumin, and 0.05% (v/v) Tween −20, and then sprayed with a sprayer at 4 mm in from the right end of the nitrocellulose membrane to form the control (C) line, with the distance between the control line and test line being 5 mm. The sprayed nitrocellulose membrane was placed in a 25° C. vacuum oven to dry at constant temperature, and then stored in a dry environment at room temperature.

2) Assembly

The following were sequentially applied in an overlapping manner onto cardboard: nitrocellulose membrane, glass cellulose pads, nitrocellulose membrane marked with test and control lines, filter paper and sample pads, and absorbent paper. After full assembly, the cardboard was cut into test strips of 4 mm in width, which were kept under seal in dry plastic kegs, having a shelf life of up to a year or longer.

### Exemplary Step 8: Melamine Detection

A 100 μL milk sample was added to the nanosphere probes (containing antibody to melamine). A chromatography test strip was then inserted, so that one end of the sample pad was immersed in the liquid. Following insertion for 5 min, the fluorescence could be read on a portable fluorescence reader, and a quantitative test result obtained by comparison with a built-in standard curve. The standard curve was developed by previously running the assay against a dilution curve of melamine.

### Exemplary Step 9: Accuracy Testing

1) Melamine standard solution was added to fresh milk with zero melamine content as tested by high-performance liquid chromatography/mass spectrometry, to give solutions with melamine concentrations of 0 ng/mL, 10 ng/mL, 20 ng/mL, 40 ng/mL, 80 ng/mL, 160 ng/mL, 320 ng/mL, and 640 ng/mL. The testing method of Exemplary Step 8 was then used for assay.

2) The experiment was repeated ten times and the results given in Table 3 below.

TABLE 3

| | Added concentration (ng/mL) | | | | | | | | | |
|---|---|---|---|---|---|---|---|---|---|---|
| | 0 | 5 | 10 | 20 | 40 | 80 | 160 | 320 | 640 | 1280 |
| Actual | 0 | 1.8 | 7.6 | 18.4 | 37.3 | 74.8 | 152.2 | 304.8 | 628.6 | 812.8 |
| measured | | | | | | | | | | |
| concentration | | | | | | | | | | |
| (ng/mL) | | | | | | | | | | |

The experimental results show that for the fluorescent quantitative detection test strips according to the present invention, the limit of quantification for melamine in the samples was 10 ng/mL, and the quantitative linear range was 10-640 ng/mL, with sample recoveries all ranging between 80% and 120%, fully meeting the needs of quantitative testing. The level of sensitivity is 10 times higher than that of colloidal gold immunochromatographic strips prepared from the same antigen/antibody raw material.

### Exemplary Step 10: Sensitivity Comparison

The sensitivity of using lyophilized nanosphere probes and having nanosphere probes spotted on sample pads was compared. In Method 1, nanospheres prepared as described in Exemplary Step 4 were lyophilized in reaction bottles described in Exemplary Step 6. In Method 2, the nanosphere probes were not lyophilized but sprayed on a sample pad. The sample pad was then dried in 37° C. for 24 hours, and then attached to nitrocellulose membranes as described in Exemplary Step 7.

Reagents from Method 1 and 2 were assayed with melamine standards as described in Exemplary Step 8 and 9. Results are shown in Table 4.

TABLE 4

| | Melanine Concentration (ppb) | | | | | | |
|---|---|---|---|---|---|---|---|
| | 10 | 20 | 40 | 80 | 160 | 320 | 640 |
| Recovered by | 8.8 | 18.4 | 37.3 | 74.8 | 152.2 | 304.8 | 325.73 |
| Method 1 | | | | | | | |
| (ppb) | | | | | | | |
| Recovered by | 0.5 | 3.45 | 8.71 | 68.83 | 143.81 | 332.63 | 638.84 |
| Method 2 | | | | | | | |
| (ppb) | | | | | | | |

Results show that Method 1, which utilized lyophilized nanospheres reagents, is a more sensitive method in comparison to having the nanospheres dried in the sample pad. According to the results in Table 4, Method 1 can detect melamine down to 10 ppb, whereas Method 2 has a sensitivity limit of approximately 80 ppb (sensitivity is defined as having the capability of detecting 80-120% of the established concentration of the analyte).

FIG. 3 is a flow diagram of an exemplary method of implementing an embodiment of the invention. The method 300 begins at step 305 and continues to step 310 wherein a liquid sample is obtained. Next, at step 315 the sample is added to a vessel (bottle, test tube, etc.) containing dried stabilized nanoparticle probes and mix. At optional step 320, the vessel is then incubated in a heating block at an incubation temperature (e.g. 37° C.). Following step 320, at step 325 the test strip is introduced into the vessel and also developed for a time period (e.g. 6 minutes). Step 325 can be conducted in a heating block at a set temperature (e.g. 37° C.). The method 300 continues to step 330 where the test strip is removed from the incubator and exposed to a time resolved fluorescent reader capable of detecting and recording fluorescence at the T and C lines on the test strip. The method then ends at step 335.

The foregoing description of embodiments of the invention comprises a number of elements, devices, machines, components and/or assemblies that perform various functions as described. These elements, devices, machines, components and/or assemblies are exemplary implementations of means for performing their respectively described functions.

### Additional Embodiments

Additional embodiments include of the assay kit includes wherein the nanosphere probe comprises Eu³⁺ and another lanthanide, with the other lanthanide in a molar percentage of lanthanide of 0.1% to 10%. The other lanthanide may be Sm³⁺, Tb³⁺, Nd³⁺, Dy³⁺, or a mixture thereof. Furthermore, wherein the analyte capturing member is an antibody to melamine.

Although only a few exemplary embodiments of the present invention have been described in detail above, those skilled in the art will readily appreciate that many modifications are possible in the exemplary embodiments without materially departing from the novel teachings and advantages of this invention.

1. A method for assaying to detect the presence or quantity of an analyte in a test sample, comprising:
forming an unbounded aqueous mixture of a first test sample with a defined amount of a nanosphere probe which is conjugate of an analyte capturing member and a long emission fluorescent label;
contacting a contacting zone of a test strip with the aqueous mixture to cause flow of the aqueous mixture from the contacting zone through a test zone of the strip that is a porous membrane, the test zone including, sequentially spaced from the contacting zone, a test area and a control area,
the test area having bound thereat a test binding moiety that (a) for a competition assay binds any sample in competition with the analyte capturing member or (b) for a sandwich assay binds any sample analyte non-competitively with the analyte capturing member.
the control area having bound thereat a control binding moiety that binds nanosphere probe;
selectively measuring long emission fluorescence at the test and control areas; and
for a given test strip, determining a test zone value normalized with the total of the test and control area signals.

2. The method of claim 1, comprising a competitive assay for melamine.

3. The method of claim 1, wherein the nanosphere probe comprises Eu³⁺ and another lanthanide, with the other lanthanide in a molar percentage of lanthanide of 0.1% to 10%.

4. The method of claim 3, wherein the other lanthanide is Sm³⁺, Tb³⁺, Nd³⁺, Dy³⁺, or a mixture thereof.

5. The method of claim 1, wherein the method is a competition assay.

6. The method of claim 1, wherein the method is a sandwich assay.

7. The method of claim 1, comprising conducting the method of a second test sample using the same defined amount of nanosphere probe.

8. An assay kit comprising:
i) a test strip that comprises a test zone that is a porous membrane, the test zone including, sequentially spaced from the contacting zone, a test zone and a control zone,
the test zone having bound thereat a test binding moiety that (a) for a competition assay binds any sample in competition with the analyte capturing member or (b) for a direct assay binds any sample analyte non-competitively with the analyte capturing member.
the control zone having bound thereat a control binding moiety that binds nanosphere probe; and
ii) a vessel with a defined amount of a dried, stabilized nanosphere probe which is conjugate of an analyte capturing member and a long emission fluorescent label.

9. The assay kit of claim 8, which is an assay kit for testing for the presence of melamine, wherein the test binding moiety is a conjugate of melamine and a protein.

10. The assay kit of claim 8, wherein the nanosphere probe comprises Eu³⁺ and another lanthanide, with the other lanthanide in a molar percentage of lanthanide of 0.1% to 10%.

11. The assay kit of claim 10, wherein the other lanthanide is Sm³⁺, Tb³⁺, Nd³⁺, Dy³⁺, or a mixture thereof.

12. The assay kit of claim 10, wherein the kit comprises two or more said vessels with the same defined amount of a nanosphere probe.

13. The assay kit of claim 8, wherein the assay is adapted for a competition assay.

14. The assay kit of claim 8, wherein the assay is adapted for a sandwich assay.

15. A nanosphere probe that is conjugate of an analyte capturing member and a long emission fluorescent label, wherein the nanosphere probe comprises Eu³⁺ and another lanthanide, with the other lanthanide in a molar percentage of lanthanide of 0.1% to 10%.

16. The nanosphere probe of claim 10, wherein the other lanthanide is Sm³⁺, Tb³⁺, Nd³⁺, Dy³⁺, or a mixture thereof.

17. The nanosphere probe of claim 10, wherein the analyte capturing member is an antibody to melamine.
